(19) United States
(12) Patent Application Publication
(10) Pub. No.: US 2010/0016844 A1
(43) Pub. Date: Jan. 21, 2010

(54) TRANSILLUMINATING FOLEY CATHETER TO FACILITATE PROSTATE AND BLADDER SURGERY

(76) Inventor: Manoj B. Patel, JR., Jacksonville, FL (US)

Correspondence Address: MARK YOUNG, P.A., 12086 FORT CAROLINE ROAD, UNIT 202, JACKSONVILLE, FL, 32225, US

(21) Appl. No.: 12/176,639

(22) Filed: Jul. 21, 2008

(51) Int. Cl. A61B 18/18; A61M 25/10

(52) U.S. Cl. 606/15; 604/96.01

(57) ABSTRACT

A catheter assembly includes an elongated flexible (transparent or translucent) tube with a drainage lumen, an inflatable balloon fluidly coupled to an inflation adapter by an inflation lumen, and an illumination adapter configured to optically couple a light source to the catheter assembly. At least one illumination lumen contains at least one flexible elongated light emitting element such as an optical fiber, OLED fiber, LED strand or waveguide adapted to emit light at one or more points along the length of the flexible elongated light emitting element. The flexible elongated light emitting element emits sensible light through the bladder and through the urethra between the urethral meatus and prostate to illuminate the bladder, the bladder neck, the urethra adjacent to the prostate and surrounding tissue, thereby reducing the risk of damage to adjacent tissue. To provide various colors of light, colored light sources and/or colored filters may be utilized. To enable emission of light at determined points of an optical fiber, cladding (and jacket, if any) is removed from the optical fiber.

### FIELD OF THE INVENTION

This invention generally relates to prostate and bladder surgery, and more particularly, to a catheter with illuminated zones to facilitate visually differentiating a prostate from other tissue, including the bladder and adjacent nerves, blood vessels and tissue.

### BACKGROUND

As shown in FIGS. 1 and 2, the bladder 105 is a hollow, expandable, muscular organ located in the pelvic girdle. The internal floor of the bladder includes a triangular area called the trigone. The ureters 100 are attached to two posterior openings, called ureteral orifices. A caudal opening, at the apex of the trigone, contains a funnel-like continuation called the neck 120 of the bladder 105. The bladder neck 120 is continuous with the urethra 135.

The wall of the bladder 105 consists of bundles of smooth muscle fibers. These muscle fibers, interlaced, form the detrusor muscle (which forms the wall of the bladder 105) and comprise the internal urethral sphincter. The internal 120 and external 130 urethral sphincters prevent urine from escaping the bladder until the pressure inside the bladder reaches a certain level.

The male urethra 135 is an important organ of both the urinary and reproductive systems. The urethra 135 transports sperm through the penis 140 to outside the body. Additionally, the urethra 135 carries urine from the bladder 105 to outside of the body. The urinary meatus 145 is the external urethral orifice.

The prostate 110, made of smooth muscle and glandular tissue, surrounds the first part of the urethra nearest the neck of the bladder (also known as the posterior urethra). Prostates may vary considerably in size and shape. The prostate 110 secretes an alkaline fluid to keep sperm mobile, protecting sperm from acid secretions of the female vagina. The prostate 110 discharges this substance into the urethra 135 as part of the ejaculate during sexual stimulation.

Bulbourethral glands (also known as Cowper's glands), lying within the external urethral sphincter 130, are two pea-sized bodies located below the prostate gland and lateral to the urethra. These glands 130 release a mucous-like fluid in response to sexual stimulation and provide lubrication to the end of the penis in preparation for sexual intercourse.

Neurovascular bundles 115, 125 comprise nerves, arteries, veins and lymphatics that travel together in close proximity to the prostate. Among the nerves are cavernous nerves, which facilitate penile erection.

The penis 140 is a cylindrical organ that conveys urine and semen via the urethra 135 through the meatus 145 to the outside. The penis is made up of three separate cylinders; two paired cylinders called the corpora cavernosa and the third cylinder called the corpus spongiosum (which contains the urethra). These three cylinders are encased in a thick tough membrane called Buck's fascia. Each of the corpora cavernosal cylinders is encased in a very tough thick sheath called the tunica albuginea.

The testes 205 are oval glands suspended inside a sac, i.e., the scrotum 200. The epididymis 210 is a convoluted tubule connecting efferent ducts from the testes to the vas deferens 235, a tube that connects the epididymis 210 and ejaculatory duct. The vas deferens 235 and the seminal vesicles 220 converge, just before the entrance of the prostate gland 110, to form the ejaculatory ducts. The seminal vesicles 220 are two pouches that merge with the vas deferens 235 near the base of the urinary bladder 105. During ejaculation, the contents of the seminal vesicles 220 are emptied into the prostatic urethra via the ejaculatory ducts.

For further perspective, skeletal and rectal features are also shown. The anus 215 is the external opening of the rectum, which leads to the colon 230. The coccyx 225 or tailbone is illustrated behind the colon 230. The pubic symphysis 240 is a midline cartilaginous joint anterior to the bladder 105 and superior to the external genitalia.

Surgical intervention for prostate cancer typically involves a radical prostatectomy via an abdominal (retropubic) or perineal approach. A radical prostatectomy is a surgical procedure in which the prostate is removed. By way of illustration, in a retropubic prostatectomy an incision is made between the umbilicus and the top of the pubic bone.

One major source of potential bleeding during prostatectomy is the dorsal vein complex 121, 122 that passes over the anterior surface of the prostate gland 110 to enter the penis 140. Thus, to control bleeding, the dorsal vein complex 121, 122 is usually tied off with sutures, i.e. suture-ligated. These sutures limit back bleeding on transection of the dorsal vein complex 121, 122.

In order to remove the prostate 110, a portion of the posterior urethra 135 is removed along with the prostate 110 gland. The prostate 110 and surrounded portion of the urethra 135 are removed below (distal) the neck 120 of the bladder 105, which is palpable during open surgery. The bladder 105 and remaining portion of the prostatic-membranous urethra 135 are then reconnected over a Foley catheter via sutured reanastomosis. A thin catheter (e.g., Foley catheter) is inserted through the penis into the bladder 105 to drain urine and allow the connection to heal. The catheter remains in place for about a week or two, or until the new connection of bladder 105 to urethra 135 has completely healed.

If cancer is clinically unlikely to have spread beyond the prostate 110, a nerve-sparing procedure may be performed to minimize impotency and speed up urinary control (continence). A nerve-sparing radical prostatectomy protects the cavernous nerves within the penis 140 which control erection. Injury to these nerves during surgery may lead to impotence. These nerves exit the penis as the neurovascular bundle 115, 125 which run adjacent to the prostate 110 as it courses in a posterior-lateral fashion to the pelvic plexus.

In recent years, robotic prostatectomy operations have become popular as less invasive alternatives for removal of the malignant prostate gland. To perform the robotic operation, the surgeon makes several small incisions through which robotic instruments are placed. The surgeon then manipulates the instruments to perform the necessary steps, including removal of the prostate 110 and surrounded portion of the urethra 135 and subsequent attachment of the membranous urethra 135 to the bladder neck 120.

Unfortunately, the bladder neck 120, blood vessels and neurovascular bundles 115, 125 are susceptible to injury during the radical prostatectomy procedure. The risk of injury is particularly high in a robotic or laparoscopic prostatectomy, because visibility is limited and the surgeon cannot detect the margins of a prostate 110 by touch (i.e., tactile sense). Furthermore, prostates 110 vary considerably in shape and size, making the locations for incision difficult to predict with any meaningful degree of accuracy, especially at the bladder neck 120-prostate 110 junction and the prostate 110-membraneous urethra 135 junction, near the external urethral sphincter 130. A slightly misplaced incision risks unintended damage to the bladder 105, blood vessels and/or nerves. Such damage may result in incontinence, erectile dysfunction, and other complications. More importantly, some tumor may be left behind in the patient during prostate 110 cancer resection surgeries, resulting in positive surgical resection margins.

What is needed is a means for distinguishing the prostate 110 from surrounding tissue, nerves and blood vessels during surgery, including robotic and laparoscopic procedures. The invention is directed to overcoming one or more of the problems and solving one or more of the needs as set forth above.

### SUMMARY OF THE INVENTION

To solve one or more of the problems set forth above, in an exemplary implementation of the invention, a catheter assembly with illuminated zones to facilitate visually differentiating a prostate from other tissue, including the bladder neck and adjacent neurovascular bundles and dorsal venous complex from the membranous urethra is provided. The catheter assembly includes an elongated flexible tube with a perforated proximal end and an open outlet defining a distal end. A drainage lumen extends from and fluidly couples the perforated proximal end to the open outlet. An inflatable balloon is attached to the elongated flexible tube adjacent to the perforated proximal end. An inflation adapter is adjacent to the distal end of the elongated flexible tube. An inflation lumen extends from and fluidly couples the inflation adapter to the inflatable balloon. An illumination adapter adjacent to the distal end of the elongated flexible tube is configured to optically couple a light source to the catheter assembly. At least one illumination lumen extends from an energy interface such as an illumination adapter to a point adjacent to the proximal end of the elongated flexible tube. The illumination lumen is configured to receive a light emitting means such as an optical fiber, an organic light emitting diode fiber, a light emitting diode strand or a waveguide. The elongated flexible tube is configured to transmit light emitted from within the illumination lumen (i.e., the tube and illumination lumen are translucent or transparent). At least one light emitting means such as an optical fiber, an organic light emitting diode fiber, a light emitting diode strand or a waveguide is contained in the illumination lumen. The at least one light emitting means is configured to emit light at a plurality of portions (or a continuum of portions) of the catheter, at a proximal portion of the catheter near the balloon and at an intermediate portion of the catheter between the proximal portion of the catheter and the distal end of the catheter. In one embodiment, at least two optical fibers are provided. One optical fiber is configured to emit light at a first portion. A second optical fiber is configured to emit light at a second portion. In another embodiment, an optical fiber emits light along its length. In yet another embodiment, an organic light emitting diode is provided and is configured to emit light at determined portions or along its length. In still another embodiment a light emitting diode strand is provided and configured to emit light a determined portions or along the length of the illumination lumen. In still another embodiment a waveguide is provided and configured to emit light a determined portions.

The elongated flexible tube is configured for extension from a male urethral meatus through the urethra and surrounding prostate, through the bladder neck and into the bladder. The balloon is inflated within the bladder. The at least one flexible optical fiber is configured to emit visible light within the bladder and within the entire urethra especially at the membranous urethra at the external urethral sphincter between the urethral meatus and prostate. The light illuminates the bladder the bladder neck and the urethra proximal to and distal to the prostate. The illumination facilitates identification of the margins of the prostate bladder neck and urethra thereby reducing the risk of damage to adjacent tissue.

To further facilitate identification, the light may be white light or light of one or more determined colors (e.g., wavelengths), such as yellow, blue and green. All light emitters may emit the same color light. Alight emitter may be configured to emit light having a first color at a first portion of the light emitter and light having a second color at a second portion of the light emitter. Illustratively, an optical fiber may be configured to emit light having a first color at a first portion (e.g., within the bladder to illuminate the bladder including the bladder neck, while another optical fiber may be configured to emit light of a second color at a second portion (e.g., within the urethra between the prostate and the external urinary sphincter at the genitourinary diaphragm).

To provide various colors of light, colored light sources and/or colored filters may be utilized. One or more color filters may be contained within the illumination adapter and provide an optical coupling between the light source(s) and optical fiber(s). Alternatively, one or more color filters may be placed over the portions of the optical fiber through which light is emitted. As another alternative, the light emitters may be colored or tinted to emit a desired color light.

The optical fibers are comprised of an optical fiber core surrounded by a cladding layer. To enable emission of light being transmitted through the core, some cladding is removed. Specifically, the cladding is partially or fully removed, either mechanically (e.g., abraded) or chemically removed (e.g., etched), at the portions of the optical fiber at which light will be emitted. With partial removal, appreciable light will continue to be internally reflected and transmitted along the remaining length of the fiber.

In lieu of an optical fiber, other flexible elongated light emitters may be used, including (without limitation) an organic light emitting diode fiber, a light emitting diode strand and/or a waveguide. In the case of an organic light emitting diode fiber or a light emitting diode strand, a power supply provides electrical energy to the flexible elongated light emitters.

### BRIEF DESCRIPTION OF THE DRAWINGS

The foregoing and other aspects, objects, features and advantages of the invention will become better understood with reference to the following description, appended claims, and accompanying drawings, where:

FIG. 1 shows a schematic front view of an exemplary illuminated catheter inserted into a bladder 105 through a urethra 135 in accordance with principles of the invention; and

FIG. 2 shows a schematic side view of an exemplary illuminated catheter inserted into a bladder 105 through a urethra 135 in accordance with principles of the invention; and

FIG. 3 shows a perspective view of an exemplary un-inserted and un-inflated illuminated catheter in accordance with principles of the invention; and

FIG. 4 shows a cross section view of an exemplary tube with a central channel, an inflation lumen, and several illumination lumens for an illuminated catheter in accordance with principles of the invention; and

FIG. 5 shows a cross section view of an exemplary tube with a central channel, an inflation lumen, and several illumination channels for an illuminated catheter in accordance with principles of the invention; and

FIG. 6 shows a perspective view of portions of an exemplary optical fiber with a modified portion of cladding layer and color filter configured to transmit diffuse colored light radially outward for an illuminated catheter in accordance with principles of the invention; and

FIG. 7 shows a perspective view of portions of an exemplary optical fiber with a color filter inlet and a modified portion of cladding layer configured to transmit diffuse colored light radially outward for an illuminated catheter in accordance with principles of the invention; and

FIG. 8 shows a perspective view of an exemplary optical fiber with a modified portion of cladding layer configured to transmit diffuse light radially outward in two distinct zones for an illuminated catheter in accordance with principles of the invention; and

FIG. 9 shows a perspective view of an exemplary optical fiber with a modified portion of cladding layer configured to transmit diffuse light radially outward for an illuminated catheter in accordance with principles of the invention; and

FIG. 10 shows a perspective view of layered portions of an exemplary OLED fiber configured to transmit light radially outward for an illuminated catheter in accordance with principles of the invention; and

FIG. 11 shows an LED strand configured to transmit light radially outward in a proximal zone for an illuminated catheter in accordance with principles of the invention; and

FIG. 12 shows a plurality of exemplary terminal end configurations for emitting light in a determined manner from optical fibers in accordance with principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the types of components, shapes, relative sizes, ornamental aspects or proportions shown in the figures.

### DETAILED DESCRIPTION

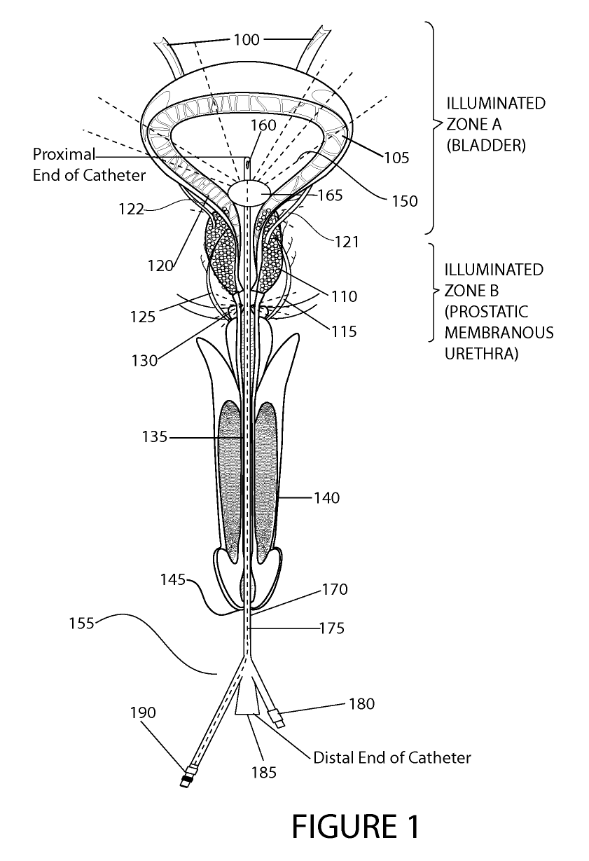

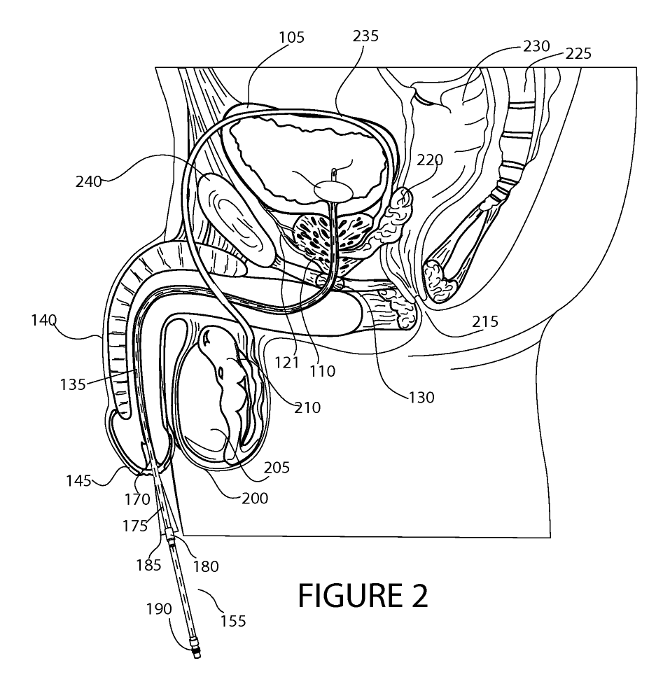

Referring to the Figures, in which like parts are indicated with the same reference numerals, various views of components and an exemplary catheter assembly with illuminated zones to facilitate visually differentiating a prostate 110 from other tissue according to principles of the invention are shown. With reference to FIGS. 1 and 2, the exemplary catheter is a Foley-style catheter modified to incorporate one or more optical fibers 175 and provide an additional arm 190 for operably coupling a light source to the distal (i.e., receiving) ends of the optical fibers. The catheter comprises an elongated, soft, flexible tube 170 (e.g., latex, silicon, polyvinylchloride, polytetrafluoroethylene or other biocompatible elastomeric tube) that is passed through the urethra 135 and into the bladder 105 to drain urine during urinary catheterization. The tube 170 is transparent or translucent. The tube 170 is retained by means of a balloon 165 near the perforated tip 160. The balloon 165 is normally deflated until properly positioned in a patient's bladder 105. Once the catheter is properly positioned, inflation fluid (e.g., sterile water) is delivered via syringe 300 into a valve assembly 180 through an inflation lumen to inflate the balloon 165. The inflated balloon 165 holds the catheter in place and impedes unintended withdrawal through the bladder 105 and bladder neck 120. The balloon may come in several different sizes such as 10 cc, 30 cc and 60 cc; although, the invention is not limited to any particular size.

The outer diameter of the tube 170 is small enough to fit within the urethra 135 and large enough to house internally contained components and lumens and to facilitate evacuation of urine from the bladder 105. The relative size of the tube in French gauge units (F) is approximately 10 F to 28 F, with 10 F being equivalent to 3⅓ mm in diameter and 18 F being equivalent to 6 mm in diameter. However, the invention is not limited to any particular size.

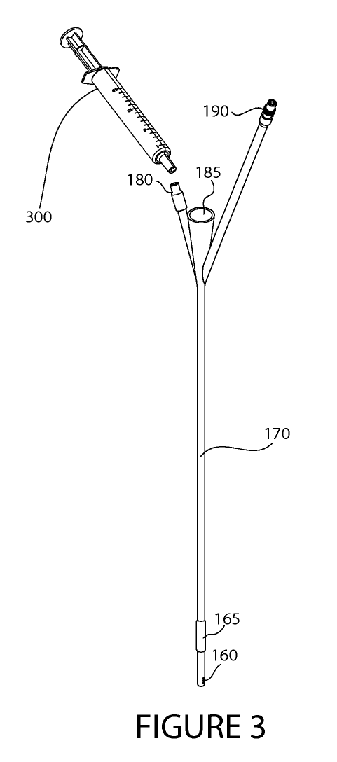

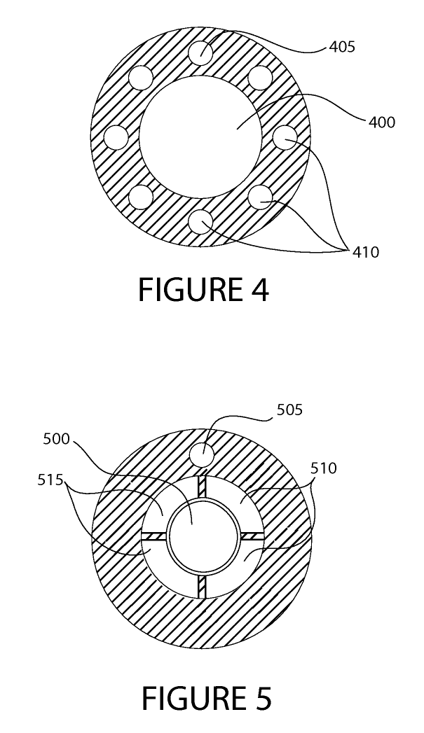

The catheter 155 includes an inflation lumen 405, 505, as shown in the cross-sectional views of FIGS. 4 and 5, and an inflation adapter 180, as shown in FIGS. 1, 2 and 3. The inflation lumen 405, 505 is a fluid-transporting channel that extends from the inflation adapter 180, through the tube 170 to the balloon 165. The inflation adapter 180 includes a valve, such as a spring actuated valve with a valve stem urged into a sealed position by a spring. A determined amount (e.g., 30 cc) of fluid (e.g., sterile water) is urged from a syringe 300 into the adapter 180. The fluid travels in the inflation lumen 405 from the adapter 180 through the tube 170 into the balloon 165, thereby inflating the balloon 165, as shown in FIGS. 1 and 2. In FIG. 3, the balloon 165 is shown deflated for insertion into a patient's bladder 105 through the urethra 135.

The catheter 155 includes a drainage lumen 400, 500, as shown in the cross-sectional views of FIGS. 4 and 5, and an outlet 185, as shown in FIGS. 1, 2 and 3. The drainage lumen 400, 500 is a fluid-transporting channel that extends from the outlet 185, through the tube 170 to the perforated proximal end 160. When the catheter 155 is inserted into the bladder 105, urine flows from the bladder 105 into the perforated proximal end 160, through the drainage lumen 400 and out of the outlet 185. The outlet may be fluidly connected to a drainage container such as a bag to collect the urine.

In addition to a drainage lumen and an inflation lumen as typically found in Foley catheters, the exemplary catheter also includes one or more illumination lumens 410, 510, 515, as shown in the cross-sectional views of FIGS. 4 and 5, for receiving one or more flexible illumination means, such as one or more optical fibers coupled to an illumination adapter 190, or an organic light emitting diode fiber as described in U.S. Pat. No. 6,538,375 (the entire contents of which are incorporated herein by this reference) coupled to a power supply, or a strand of miniature light emitting diodes coupled to a power supply. The illumination lumen 410, 510, 515 is a channel that extends from the illumination adapter 190, through the tube 170 to either the balloon 165 or perforated proximal end 160. When the catheter 155 is inserted into the bladder 105, a light emitting means illuminates the catheter 155. By way of example and not limitation, a light source may supply visible light to the illumination adapter 190. The illumination adapter 190 optically couples the optical fiber contained in the illumination lumen 410, 510, 515 to the light source. In a preferred embodiment, the adapter 190 is a plug that fits standard surgical light sources, such as A.C.M.I. MV 9082, MV 9083 and ALV-1 light sources, Olympus® surgical light sources and Karl Storz® surgical light sources, with or without the use of adapter couplings. The optical fiber contained in the illumination lumen 410, 510, 515 transmits light from the light source to one or more illumination zones to illuminate the bladder 105 and urethra 135 from within at determined zones, such as zones A and B as conceptually shown in FIG. 1. While the prostate 110 gland is generally opaque, the bladder 105 and urethra 135 are translucent. Light 150 will pass through the illuminated bladder 105 and urethra 135 to enhance visibility of the prostate 110 margins and surrounding tissue. The less illuminated prostate 110 margins are visibly differentiated from the highly illuminated bladder 105, including the bladder neck 120 and the membranous urethra 130. Likewise, the neurovascular bundles and cavernous nerves are illuminated from light emanating from the illuminated urethra 135.

The illumination adapter 190 is a fiber-optic connector, e.g., a rigid cylindrical barrel surrounded by a sleeve that holds the barrel in a mating socket. A mating light source can be “push and click”, “turn and latch” (i.e., “bayonet”), or screw-in (i.e., threaded).

Light emitting means other than an optical fiber may be utilized and are intended to come within the scope of the invention. By way of example and not limitation, an organic light emitting diode fiber coupled to a power supply, or a strand of miniature light emitting diodes coupled to a power supply may be utilized in lieu of the optical fiber.

The term light can have various meanings, but here it is used to refer to the portion of the electromagnetic spectrum with vacuum wavelengths λ_{vac} in the range of 1 μm to 100 nm (or in the frequency v=c/λ, 3×10¹⁴ to 3×10¹⁵ Hz, where c is the speed of light). This spectral range includes the near infrared (NIR), the visible, and the ultraviolet (UV) A, B, and C bands. Infrared and ultraviolet light not perceptible by the naked eye, may readily be perceived using compatible surgical imaging equipment.

Sensible light emitted from a catheter according to principles of the invention will penetrate and thereby illuminate the urethra and bladder. This illumination is readily sensible by the naked eye and/or using compatible surgical imaging equipment. Such equipment may be configured to generate an image based on visible light, infrared light and/or UV light emitted from the catheter. If light is emitted from a prostatic urethral portion of the catheter, the prostate will absorb most or all of the emitted light, making any illumination of the prostate non-existent or extremely faint. The faint or non-illumination of the prostate in contrast to the illuminated bladder, bladder neck and urethra portions adjacent to the prostate visibly defines the margins of the prostate. Light emitted from the bladder, bladder neck and urethra portions adjacent to the prostate also illuminate surrounding tissue.

As discussed above, a catheter according to principles of the invention requires drainage, optical fiber and inflation lumens. However, the particular arrangement and configuration of lumens is not particularly important so long as each lumen provides the requisite functionality. Referring now to FIG. 4, a cross-section conceptually illustrating an alternative arrangement of lumens is provided. The inflation lumen 405 links the inflation adapter 180 to the balloon 165. The drainage lumen 400 provides a fluid-transporting channel that extends from the outlet 185, through the tube 170 to the perforated proximal end 160. The illumination lumens 515, 510, 410 comprise channels that extend from the illumination adapter 190, through the tube 170 to either the balloon 165 or perforated proximal end 160.

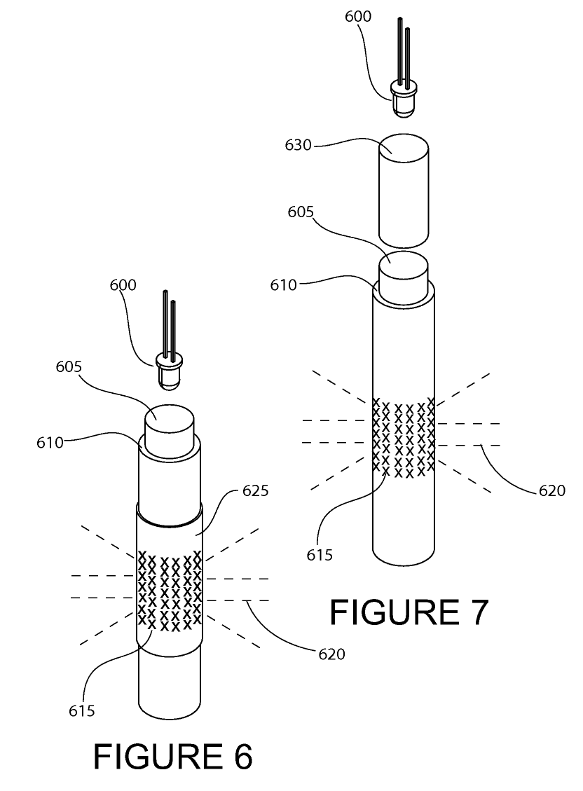

An optical fiber 175, exemplary portions of which are conceptually illustrated in FIGS. 6 and 7, carries light from a light source 600 along its length. The optical fiber 175 is elongated and flexible, extending substantially the length of the catheter and allowing the fiber 175 to conform to the shape of the tube 170 as it bends during insertion, removal and use. The optical fiber 175 is comprised of a glass or plastic core 605 surrounded by a cladding layer 610. Optionally, a jacket layer (not shown) may cover the cladding layer. Light is maintained in the core 605 of the optical fiber 175 by total internal reflection, causing the fiber to act as a light guide. Using the optical fiber, bright light may be transmitted from a light source 600 to targeted areas of the urethra 135 and bladder 105 without a clear line-of-sight path. A plurality of optical fibers 175 can be bundled as light emitting cables.

In a particular embodiment, one or more optical fibers 175 emit a specific color light 620A for zone A and a different color light 620B for zone B. Preferably the colors are not naturally found within a body. For example, blue, green and yellow colors may be used. The colors may be produced using one or more colored light sources, tinting and/or filters. A colored light source 600 may be utilized for an optical fiber 175 to emit a specific color light. A plurality of colored light sources 600 may be provided for a plurality of optical fibers 175 to each emit a different color light. A colored light filter 630 may be disposed between a light source 600 and a core 605 of an optical fiber 175. A colored filter sleeve 625 or coating may cover the light emitting portions 615 of an optical fiber 175. Light that passes through the sleeve 625 or filter 630 takes on the color of the filter. Alternatively, the optical fiber 175 may be tinted to emit a determined color light.

A light source 600, such as a light emitting diode (LED), is provided for illumination. The LED 600 emits visible light when a current passes through it in the correct direction. The color, size, shape, and viewing angle of the LED may be selected to achieve satisfactory visibility in a compact, lightweight, energy efficient design. A miniature (e.g., 5 mm round cross-section) or subminiature (e.g., 3 mm round cross section) LED with a 30° or 60° viewing angle may be utilized. A lens, transparent cover or aperture may be provided to promote and/or enable transmission of light from the light source 600 to the optical fiber 175.

A light emitting finish is formed on portions 615 of the outer surface (i.e., cladding 610) of the optical fibers 175 to transmit light (e.g., diffuse light emitted radially outward). In particular, the terminal end may emit light and/or cladding (and any jacket layer) may be partially removed to emit diffuse light. As used herein, partial removal refers to any mechanical, thermal and/or chemical treatment that causes the optical fibers 175 to diffusely emit transmitted light radially.

An exemplary process for achieving a light emitting finish entails abrasion of the optical fiber cladding. By way of example and not limitation, an abrasive material may remove cladding from the targeted areas of the optical fibers 175. Removal of some, but not all, of the cladding over a portion of the optical fiber, prevents total internal reflection. Where the cladding is removed, some light will be emitted rather than reflected into the core.

Other surface treatment methods to achieve a light emitting finish at determined portions along the length of the fiber may be utilized. Such other methods may entail chemical etching, other processes for grinding and/or roughening the surface, such as sandblasting; or processes for coating the surface with a film or other material designed to negate the reflectance property of the cladding; or processes for altering the composition of the cladding to affect the optical properties; or any other method that allows some light to be emitted from the core at the determined portions.

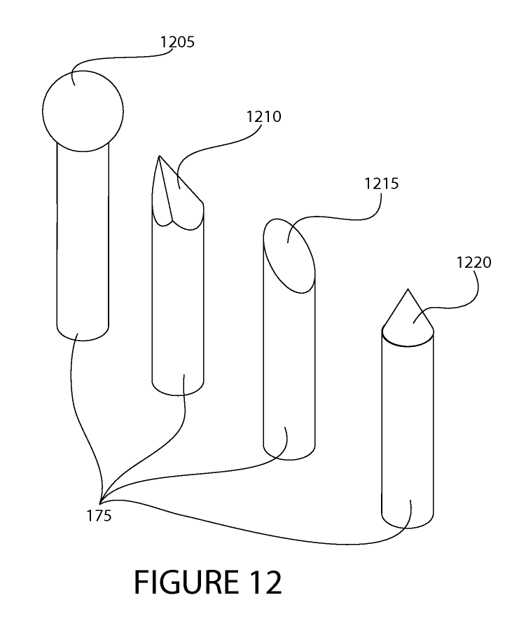

In another embodiment, the beam shape at the fiber's exit is controlled using certain types of terminal ends, i.e., tips 1205-1220, as illustrated in FIG. 12. The tips can be made by polishing the fiber materials (in the case of solid-core fiber) or from optically compatible materials adhered to the fiber 1205-1220. By way of example and not limitation, a ball shaped 1205, multiplanar tapered 1210 or conical 1220 tip may be used to diffuse light to surrounding targeted areas. As another example, a side firing tip 1215 may be used if illumination along a particular side is preferred. Thus, a plurality of optical fibers 175 may emit light at each of the targeted areas using such terminal ends.

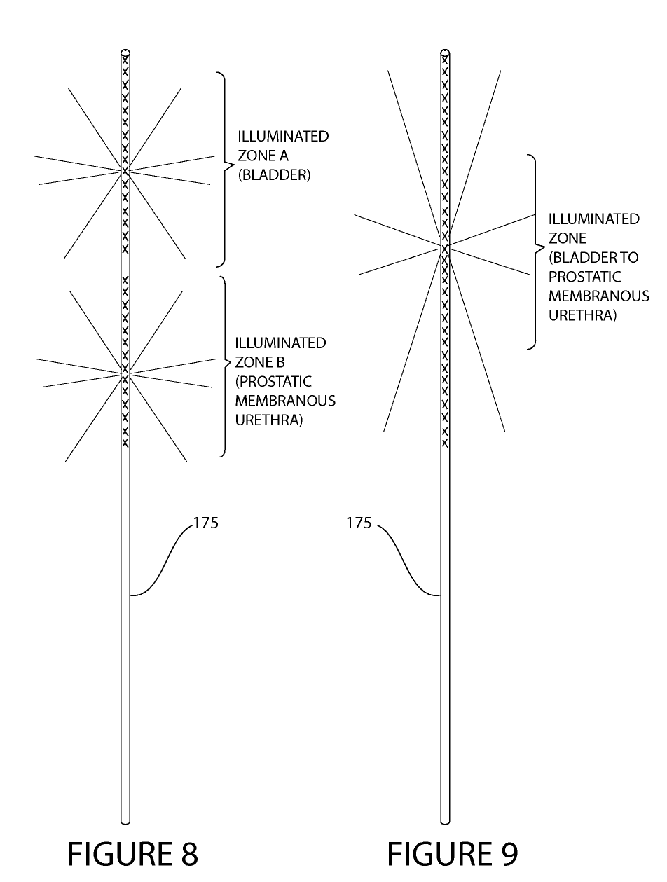

Referring now to FIG. 8, determined portions of the optical fiber that have a light emitting finish correspond to Zones A and B as shown in FIG. 1. Thus, a proximal portion of the fiber from the proximal end (i.e., the end inserted into the bladder 105) to approximately one 0.25 to 2.0 inches from the balloon towards the distal end may have a light emitting finish and define illuminated Zone A. Light emitted from this portion of the fiber will illuminate the translucent bladder 105 and bladder neck 120 and, to a lesser intensity, the prostate 110. A portion of a fiber extending from the distal end of the proximal portion, as described above, and ending approximately 0.5 to 3.0 inches from that point towards the distal end may have a light emitting finish and define illuminated Zone B. Light emitted from this portion of the fiber will illuminate the translucent prostatic-membranous urethra 135 and neurovascular bundle 115, 125 to a greater degree than the prostate 110, which will illuminate at a lesser degree. Other light emitters (e.g., an LED strand, an OLED fiber and a waveguide) may be configured to illuminate these same portions of the catheter in accordance with principles of the invention.

Referring now to FIG. 9, a determined proximal portion of the optical fiber has a light emitting finish. Thus, a proximal portion of the fiber from the proximal end (i.e., the end inserted into the bladder 105) to approximately ten 10 to 15 cm from the balloon towards the distal end may have a light emitting finish and define a continuous illuminated zone. Light emitted from this portion of the fiber will illuminate the translucent bladder 105 and bladder neck 120 and, to a lesser intensity (or not at all), the prostate 11O. Light emitted from this portion of the fiber will also illuminate the translucent prostatic-membranous urethra 135 and neurovascular bundle 115, 125 to a greater degree than the prostate 110, which will illuminate at a lesser degree. Other light emitters (e.g., an LED strand, an OLED fiber and a waveguide) may be configured to illuminate these same portions of the catheter in accordance with principles of the invention.

One or more fibers may be configured to illuminate both zones. Fibers may be configured to illuminate a single zone or a plurality of zones. Thus one fiber may be configured to illuminate Zone A, while another fiber may be configured to illuminate Zone B. The transparent tube 170 includes one or more illumination lumens 410 configured to carry one or more optical fibers. Alternatively, a fiber may be configured to illuminate both Zone A and Zone B. As another alternative, one fiber may be configured to illuminate Zone A, Zone B, as well as any space therebetween. Other light emitters (e.g., an LED strand, an OLED fiber and a waveguide) may be configured to illuminate these same portions of the catheter in accordance with principles of the invention.

Using colored filters, tinting or colored light sources, the light emitted from each zone may be a determined color. Thus, for example, light of one color (e.g., yellow) may be emitted at Zone A, while light of another distinguishable color (e.g., blue or green) may be emitted at Zone B.

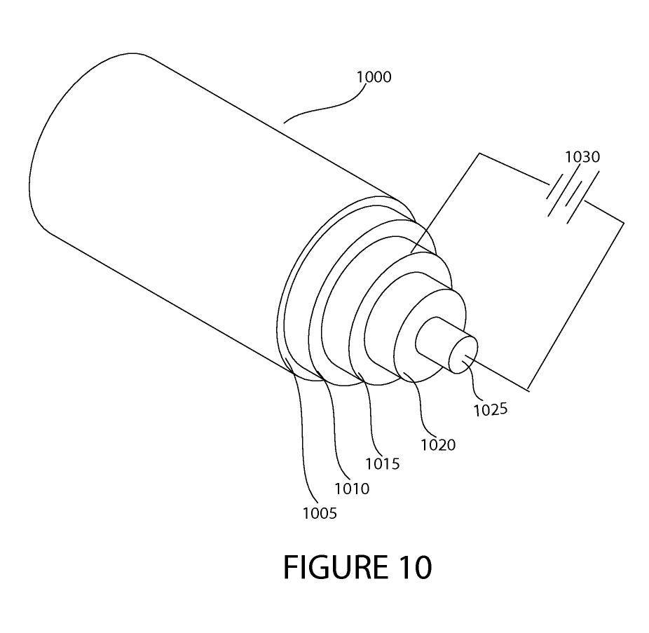

As discussed above, light emitting means other than an optical fiber may be utilized and are intended to come within the scope of the invention. By way of example and not limitation, an organic light emitting diode fiber 1000 coupled to a power supply 1030 via an illumination adapter, or a strand of miniature light emitting diodes 1100 coupled to a power supply 1150 via an illumination adapter may be utilized in lieu of the optical fiber. Referring now to FIG. 10, an exemplary organic light-emitting diode fiber 1000 comprises a core 1025 with a first electrically conducting material forming a first electrode; at least one layer of an organic electroluminescent material 1020 formed over and in direct or indirect contact with the first electrode; at least a layer of a second electrically conducting material forming a second electrode, the second electrode in a shape of a second electrode layer being formed over at least a portion of and in direct or indirect contact with the at least one layer of organic electroluminescent material 1015; and at least one barrier layer formed over the second electrode layer surrounding the organic electroluminescent material and the second electrode, the at least one barrier layer comprising a plurality of sublayers 1010, 1005 of a polymeric material and an inorganic material. The flexible core may comprise a flexible polymeric or metallic material. Suitable polymeric materials for fiber core member are polyolefins such as polyethylene, polypropylene, or polytetrafluoroethylene; polysiloxane; epoxy; polyacrylate; polyethyleneterephthalate; and derivatives thereof. A fiber core element may comprise a glass or a metal such as aluminum, copper, or steel. The core member may have a diameter of about 1 micrometer to about 2 mm, preferably about 10 micrometers to about 2 mm, and more preferably about 100 micrometers to about 1 mm. When a voltage is applied across electrodes layers of the OLED fiber, charge carriers (i.e., electrons and holes) are injected into organic electroluminescent layer, where the charge carriers recombine to form excited molecules which emit radiation when they decay to lower-energy states. Typically, the applied voltage is in the range from about 2 to about 10 V. One or more switching elements may be provided to control illumination of light elements. The thickness of electrode layers is typically in the range from about 50 nm to about 500 nm, preferably from about 50 nm to about 200 nm. Materials suitable for use as a cathode are K, Li, Na, Mg, La, Ce, Ca, Sr, Ba, Al, Ag, In, Sn, Zn, Zr, alloys thereof, or mixtures thereof. Preferred materials for the manufacture of cathode layer are Ag—Mg, Al—Li, In—Mg, and Al—Ca alloys. Indium tin oxide (“ITO”) may be used for the anode because ITO is substantially transparent to light transmission and allows at least 80% light transmitted therethrough. Therefore, light emitted from organic electroluminescent layer can easily escape through an ITO anode layer without being seriously attenuated. Other materials suitable for use as the anode layer are tin oxide, indium oxide, zinc oxide, indium zinc oxide, and mixtures thereof. The anode may also be made of a thin metal layer such as a layer of Pt, Pd, Ag, or Au, with the thickness of anode layer preferably kept such that light emitted from organic electroluminescent layer is not seriously attenuated as it travels through anode layer. Electrode layers and may be deposited on the underlying element by physical vapor deposition, chemical vapor deposition, or sputtering.

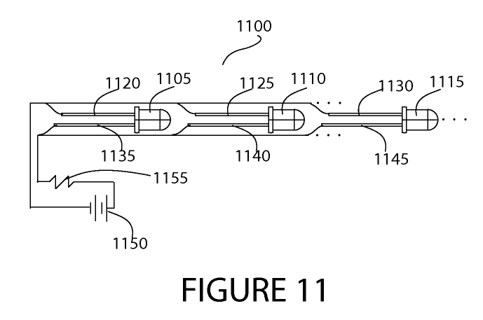

With reference now to FIG. 11, a flexible light emitting strand 1100 is shown. The strand 1100 includes a plurality of lighting elements (e.g., LEDs, such as 2 mm diameter mini LEDs) 1105, 1110, 1115, which may be connected in parallel (as shown) or in series. All cathodes 1120, 1125, 1130, anodes 1135, 1140, 1145 and electrical lines are preferably covered with a flexible insulator. A power supply 1150 such as a disposable or rechargeable battery or a power transformer coupled to a utility power supply is provided to supply electrical energy to the lighting elements. In such an embodiment, the illumination adapter comprises an electrical coupling for energizing the strand. One or more switching elements may be provided to control illumination of light elements. One or more current limiters 1155 protect the LEDs 1105, 1110, 1115 from an over current condition and conserve battery power. Current limiters 1105, 1110, 1115 may be comprised of resistors that limit the current supplied. Current limiters 1155 could be comprised of active electronic current control circuits or devices that are controlled by a microcontroller output signal. There could be one or more Current limiters 1155 per LED 1105, 1110, 1115 channel, depending upon the LED 1105, 1110, 1115 configuration.

Any other means for transmitting light that is flexible and configurable to fit within an illumination lumen of a catheter according to principles of the invention may be utilized. By way of example and not limitation, a flexible waveguide or light pipe may used to transmit light from a light source connected via an adapter, through an illumination lumen to a targeted area. The waveguide (or light pipe) comprises a hollow flexible tube internally coated with a thin reflective layer. The reflective material constrains and guides the propagation of light along the length of the waveguide to a terminal end. The terminal end may be configured with an emitting tip or lens configured to diffuse transmitted light to surrounding targeted areas.

Although the invention has been described with reference to a specific embodiment, the foregoing description is not intended to be construed in a limiting sense. Various modifications to the disclosed embodiment as well as alternative applications of the invention will be suggested to persons skilled in the art by the foregoing specification and illustrations. It is therefore contemplated that the appended claims will cover any such modifications, applications or embodiments as fall within the true scope of the invention.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

1. A catheter assembly comprising an elongated flexible tube with a perforated proximal end and an open outlet defining a distal end, a drainage lumen extending from and fluidly coupling said perforated proximal end to said open outlet, an inflatable balloon attached to said elongated flexible tube adjacent to the perforated proximal end, an inflation adapter adjacent to the distal end of the elongated flexible tube, and an inflation lumen extending from and fluidly coupling said inflation adapter to said inflatable balloon, an illumination lumen extending from said illumination adapter to a point adjacent to the proximal end of the elongated flexible tube, said illumination lumen being configured to receive an elongated flexible light emitter, said elongated flexible tube being configured to transmit light emitted from within the illumination lumen.

2. A catheter assembly according to claim 1, further comprising an elongated flexible light emitter within said illumination lumen, said elongated flexible light emitter being an elongated flexible light emitter from the group consisting of at least one flexible optical fiber, at least one organic light emitting diode fiber, at least one light emitting diode strand and at least one waveguide.

3. A catheter assembly according to claim 1, said elongated flexible light emitter comprising at least one flexible optical fiber configured to emit light at a plurality of portions of the optical fiber.

4. A catheter assembly according to claim 2, said elongated flexible light emitter comprising at least two optical fibers, a first of said at least two optical fibers being configured to emit light at a first portion of the elongated flexible tube, and a second of said at least two optical fibers being configured to emit light at a second portion of the elongated flexible tube.

5. A catheter assembly according to claim 2, said elongated flexible light emitter comprising an optical fiber configured to emit light having a first color at a first portion of the elongated flexible tube.

6. A catheter assembly according to claim 2, said elongated flexible light emitter comprising configured to emit light having a first color at a first portion of the elongated flexible tube and light having a second color at a second portion of the elongated flexible tube.

7. A catheter assembly according to claim 2, said elongated flexible light emitter being configured to emit light from the proximal end of the elongated flexible tube to a point on the elongated flexible tube between the distal and proximal ends.

8. A catheter assembly according to claim 2, said elongated flexible light emitter being configured to emit light from the proximal end of the elongated flexible tube to at least an intermediate point on the elongated flexible tube between the distal and proximal ends corresponding to the distal aspect of the prostatic urethra.

9. A catheter assembly according to claim 3, said at least one flexible optical fiber comprising an optical fiber configured to emit light at a proximal portion of the optical fiber near the balloon and at an intermediate portion of the optical fiber between the proximal portion of the optical fiber and the distal end of the elongated flexible tube.

10. A catheter assembly according to claim 1, said elongated flexible tube comprising a light transmitting material from the group comprising a transparent material and a translucent material.

11. A catheter assembly according to claim 1, said elongated flexible tube comprising a light transmitting material from the group comprising a transparent material and a translucent material, and said illumination lumen comprising a light transmitting material from the group comprising a transparent material and a translucent material.

12. A catheter assembly according to claim 2, said elongated flexible tube being configured for extension from a male urethral meatus, through a urethra and surrounding prostate and into a bladder, said balloon being configured to inflate within said bladder, and said at least one flexible optical fiber being configured to emit sensible light within the bladder and within the urethra between the urethral meatus and prostate.

13. A catheter assembly according to claim 3, said elongated flexible tube being configured for extension from a male urethral meatus, through a urethra and surrounding prostate, through a bladder neck and into a bladder, said balloon being configured to inflate within said bladder, and said at least one flexible light emitter being configured to emit sensible light within the bladder and within the urethra between the urethral meatus and prostate, said sensible light visibly illuminating the bladder, the bladder neck and the urethra adjacent to the prostate.

14. A catheter assembly according to claim 3, said at least one flexible optical fiber comprising an optical fiber configured to emit light at a proximal portion of the optical fiber near the balloon and at an intermediate portion of the optical fiber between the proximal portion of the optical fiber and the distal end of the elongated flexible tube.

15. A catheter assembly according to claim 3, said illumination adapter comprising a housing containing at least one color filter in optical communication with said at least one flexible optical fiber contained in said illumination lumen, said at least one color filter causing light emitted from an optically coupled light source to become light of a determined first color.

16. A catheter assembly according to claim 3, said at least one flexible optical fiber comprising a plurality of flexible optical fibers, and said illumination adapter comprising a housing containing a plurality of color filters, each color filter optically coupling a flexible optical fiber to a light source, and each color filter causing light emitted from the light source to become light of a color corresponding to the color filter.

17. A catheter assembly according to claim 3, further comprising a color filter covering a portion of said at least one flexible optical fiber and causing light emitted from said at least one flexible optical fiber to become light of a determined first color.

18. A catheter assembly according to claim 3, said elongated flexible tube being configured for extension from a male urethral meatus, through a urethra and surrounding prostate, through a bladder neck and into a bladder, said balloon being configured to inflate within said bladder, and said at least one flexible optical fiber being configured to emit sensible light of a first color within the bladder and sensible light of a second color within the urethra between the urethral meatus and prostate, said sensible light of the first color visibly illuminating the bladder and bladder neck, and said sensible light of the second color visibly illuminating the urethra adjacent to the prostate.

19. A catheter assembly according to claim 3, said elongated flexible tube being configured for extension from a male urethral meatus, through a urethra and surrounding prostate, through a bladder neck and into a bladder, said balloon being configured to inflate within said bladder, and said at least one flexible optical fiber being configured to emit sensible light from a first light emitting portion within the bladder and from a second light emitting portion within the urethra between the urethral meatus and prostate, said sensible light visibly illuminating the bladder, the bladder neck and the urethra adjacent to the prostate, each of said at least one flexible optical fiber comprising a central fiber optic core and surrounding cladding layer, and each of said first and second light emitting portions comprising portions of said at least one flexible optical fiber with a partially removed cladding layer.

20. A catheter assembly according to claim 3, said elongated flexible tube being configured for extension from a male urethral meatus, through a urethra and surrounding prostate, through a bladder neck and into a bladder, said balloon being configured to inflate within said bladder, and said at least one flexible optical fiber being configured to emit sensible light from a first light emitting portion within the bladder and from a second light emitting portion within the urethra between the urethral meatus and prostate, said sensible light visibly illuminating the bladder, the bladder neck and the urethra adjacent to the prostate, each of said at least one flexible optical fiber comprising a central fiber optic core and surrounding cladding layer, and each of said first and second light emitting portions comprising portions of said at least one flexible optical fiber with a partially removed cladding layer removed by a surface treatment process from the group consisting of mechanical or chemical removal.
